(19) United States
(12) Patent Application Publication
(10) Pub. No.: US 2021/0389225 A1
(43) Pub. Date: Dec. 16, 2021

(54) A FLOW CYTOMETRIC DETECTION METHOD FOR LYMPHOCYTE IN IMMUNE CELLS

(71) Applicant: MINGDAO INNOVATION (BEIJING) MEDICAL-TECH CO., LTD., Beijing (CN)

(72) Inventor: Weili YAO, Beijing (CN)

(73) Assignee: MINGDAO INNOVATION (BEIJING) MEDICAL-TECH CO., LTD., Beijing (CN)

(21) Appl. No.: 17/295,655

(22) PCT Filed: Dec. 1, 2018
(86) PCT No.: PCT/CN2018/118802

§ 371 Date: May 20, 2021

(51) Int. Cl. G01N 15/14; G01N 33/533; G01N 33/577; G01N 1/38; G01N 1/40

(52) U.S. Cl. CPC G01N 15/14; G01N 33/533; G01N 1/4077; G01N 1/38; G01N 33/577

(57) ABSTRACT

The present application relates to a flow cytometric detection method for lymphocytes in immune cells. The method includes steps of: a) lymphocyte samples stained with immunofluorescent antibodies were added into the pre-cooled PBS-EDTA solution, and the cells were mixed and prepared for flow cytometry detection; b) starting up and warming up a flow cytometer system, adjusting the flow speed, then adding PBS-EDTA solution into a sample tube, and flushing a nozzle system of liquid stream; c) the sample of homogenous cells obtained in step a) is added to the sample tube and then tested. The present detection method can separate cell subpopulations, so that the analysis and detection are more accurate, and is especially suitable for detecting cell subpopulations with small number of cells, thereby saving antibodies and reagents.

### TECHNICAL FIELD

The present application relates to the field of medicines, and in particular to a flow cytometric detection method for lymphocyte in immune cells.

### BACKGROUND

The human immune system is usually divided into: immune organs and immune tissues, immune cells (phagocytes and lymphocytes), immune active substances (antibodies, lysozyme, complement, immunoglobulin, interferon, interleukin, tumor necrosis factor and other cytokines). Among them, the traditional detection of immune lymphocytes usually includes adding fluorescent antibody to 100 μl peripheral blood, incubating for 15-30 minutes, then lysing red blood cells with hemolysin, washing, adding machine buffer solution, and testing with flow cytometer.

CN201680006060.1 discloses a method for examining an immune state, which is performed based on the ratio of each subgroup of immune cells: T, B, NK, NKT and T cells (subdivided into CD4-+T cells and CD8+T cells). This invention has the following technical shortcomings: 1. there is a waste of antibody and reagent materials; 2. there is no definition of DC cell subtype having innate immunity; 3. the definition of individual lymphocyte subpopulations is not comprehensive, and the function of lymphatic immune system is not considered systematically, so that the division of subpopulations is not comprehensive and balanced; 4. lysing red blood cells by using red blood cell lysis solution after dyeing immunofluorescent antibody results in the unclean background and too much debris, which intends to cause data errors; 5. for a small number of lymphocyte subpopulations, the method cannot detect or the results are not accurate; and 6. this method is prone to over-staining or under-staining, resulting in inaccurate data.

CN201410486089.7 discloses a method for lymphocyte immunophenotyping and a kit thereof, which further divides T cells into the same types of T(subdivided into CD4+T cells and CD8+T cells), B, NK, NKT, based on which, several T cells are defined by the combination of CD45RA and CD27, and four types of B cells are defined by CD19, CD24, CD38, and CD27. This invention has the following technical shortcomings: 1. there is a waste of antibody and reagent materials; 2. there is no definition of DC cell subtype having innate immunity; 3. the definition of individual lymphocyte subpopulations is not complete, and the function of lymphatic immune system is not considered systematically, so that the division of subpopulations is not comprehensive and balanced; 4. lysing red blood cells by using red blood cell lysis solution after dyeing immunofluorescent antibody results in the unclean background and too much debris, tending to cause data errors; 5. for a small number of lymphocyte subpopulations, the method cannot detect or the results are not accurate; and 6. this method is prone to over-staining or under-staining, resulting in inaccurate data.

Therefore, a more comprehensive and balanced method for detecting lymphocyte subpopulations is still a research hotspot, and there is still no relevant report at present.

### SUMMARY

In view of the above technical status, the present application provides a method for detecting lymphocytes in immune cells, including the steps of:

a) lymphocyte samples stained with immunofluorescence antibody were added into the pre-cooled PBS-EDTA solution, and the cells were mixed and prepared for flow cytometry detection;

b) starting up and warming up a flow cytometer, adjusting the flow speed to a low-speed range of 10-29 μl/min or a medium speed range of 30-44 μl/min, then adding PBS-EDTA solution into a sample tube, and flushing a nozzle system of liquid stream;

c) the sample of homogenous cells obtained in step a) was added to the sample tube and then tested.

In the method of the present application, as one of the embodiments, the pre-cooling temperature in step a) is a temperature commonly used for flow cytometric detection in the art, including but not limited to 2-8° C. in the present application.

In the method of the present application, as one of the embodiments, the PBS-EDTA solution in step a) or step b) is a mixed solution containing 0.005-0.05M PBS and a final concentration of 2-3 mM EDTA with pH of 7.2-7.4;

In the present application, as one of the embodiments, the 0.005-0.05M PBS, for example, can be 0.005M PBS, 0.01M PBS, 0.02M PBS, 0.03M PBS, 0.04M PBS or 0.05M PBS.

In the method of the present application, as one of the embodiments, further, the step a) further includes: the concentration of cells in the lymphocyte sample is 1×10⁶-10×10⁶/ml; the preferred range of concentration is 1.5×10⁶-5×10⁶/ml, further preferably 1.5-3.0×10⁶/ml.

In the method of the present application, as one of the embodiments, the step a) further includes: the volume of the lymphocyte sample is a volume suitable for detection, including but not limited to 50-200 μl in the present application.

In the method of the present application, as one of the embodiments, in step b), the flow speed is adjusted to a medium speed range of 30-44 μl/min; as one of the further embodiments, the flow speed is adjusted to a medium speed range of 35-40 μl/min; as an example, the flow speed can be adjusted to 35 μl/min, 36 μl/min, 37 μl/min, 38 μl/min, 39 μl/min or 40 μl/min.

In the method of the present application, as one of the embodiments, a preheating time in step b) can be the time suitable for detection, which can be adjusted by those skilled in the art, and it is preferably 5-10 minutes in the present application.

In the method of the present application, as one of the embodiments, the amount of PBS-EDTA solution added into the sample tube in step b) can be determined by those skilled in the art according to the detection equipment and conditions, for example, including but not limited to 3 ml.

In the method of the present application, as one of the embodiments, step c) further includes setting conditions as follows: 3000-20000 cells in the gate, preferably 15000 cells in the gate, and collecting samples.

In the method of the present application, as one of the embodiments, the flow cytometric cell sample is prepared by the following method:

1) in vitro anticoagulant blood samples were taken for centrifugation to separate plasma and blood cell precipitation to obtain blood cell precipitation;

2) diluent blood cell precipitation was diluted with PBS solution to obtain blood cell diluent;

3) the blood cell diluent was slowly added to a centrifuge tube containing an equal volume of lymphocyte separation solution, and then centrifuged to remove the plasma to obtain the middle buffy coat cells diluent;

4) adding the buffy coat cells obtained in step 3) into a centrifuge tube, adding PBS solution with stirring to obtain a suspension, centrifuging, removing supernatant, and then adding PBS solution to adjust the cell concentration to 1×10⁵/ml-1×10⁸/ml, preferably 1.5×10⁶/ml-5×10⁶/ml, further preferably 1.5×10⁶/ml-3.0×10⁶/ml; as an example, the concentration can be 1.5×10⁶/ml, 2.0×10⁶/ml, 2.5×10⁶/ml, 3.0×10⁶/ml;

5) adding the lymphocyte cells obtained in step 4) after adjusting the concentration thereof into a flow tube; and adding flow cytometric antibody to label lymphocyte subpopulations, mixing, standing at 2-8° C. and incubating without light;

6) adding PBS solution, mixing, and centrifuging to remove unbound antibody;

7) completion of samples

\textcircled{1} If a detection on a flow cytometer is performed immediately, the supernatant is discarded and the cells are evenly mixed with PBS-EDTA solution pre-cooled to 2-8° C. for flow cytometric detection;

\textcircled{2}If the detection cannot be performed immediately, instead of diluting with PBS solution, cells are directly resuspended by adding 0.05-5% formalin, mixed, and left to stand at 2-8° C. in the dark. Before detection, each of the tubes is added with PBS solution, centrifuged to discard the supernatant, and added with PBS-EDTA solution at 2-8° C. to mix cells for flow cytometric detection.

In the method of the present application, as one of the embodiments, the PBS solution is 0.005-0.05M PBS solution at pH 7.2-7.4; As one of the embodiments, the PBS buffer solution can contain calf serum and/or EDTA, and the concentration of the calf serum or EDTA can be the concentration commonly used in literature.

In the method of the present application, as one of the embodiments, the PBS-EDTA solution is a mixed solution at pH 7.2-7.4 containing 0.005-0.05M PBS and a final concentration of 2-3 mM EDTA.

In the method of the present application, as one of the embodiments, step 1) further includes centrifuging conditions of 1500-3500 rpm for 5-30 min, preferably 1500-2900 rpm for 15-20 min.

In the method of the present application, as one of the embodiments, step 2) further includes diluting blood cell precipitate with PBS solution by a volume ratio of 1:0.5 to 1:2, preferably 1:1.

In the method of the present application, as one of the embodiments, the lymphocyte separation solution in step 3) can be prepared by those skilled in the art according to the existing technology, or can be commercially purchased. The lymphocyte separation solution from GE company, an American reagent company, is preferred in the present application.

In the method of the present application, as one of the embodiments, step 3) further includes centrifuging conditions of 1500-3500 rpm, 10-20 min and 4° C.; preferably, 1500-2900 rpm, 15-20 min and 4° C.; in the method of the present application, as one of the embodiments, in step 4), if necessary, an appropriate amount of suspension can be selected before centrifuging and counted with a hematology analyzer for subsequent adjustment of cells concentration.

In the method of the present application, as one of the embodiments, step 4) further includes centrifuging conditions of 1500-3500 rpm, 5-20 min and 4° C.; as one of the further embodiments, the centrifuging conditions are 1500-2900 rpm, 5-10 min and 4° C.

In the method of the present application, as one of the embodiments, PBS solution is added to the centrifuge tube in step 4) until the volume is 10 ml-15 ml.

In the method of the present application, as one of the embodiments, the amount of diluted lymphocytes obtained in step 4) and added to the flow tube in step 5) can be an amount commonly used in the art, as an example, including but not limited to 100 μl;

In the method of the present application, as one of the embodiments, as an example, the incubation time in step 5) can include, but not limited to, 10-30 minutes.

In the method of the present application, as one of the embodiments, step 6) further includes centrifuging conditions of 1500-2900 rpm for 5-10 min.

In the method of the present application, as one of the embodiments, the amount of PBS solution added in step 6) is 2 ml.

In the method of the invention, as one of the embodiments, the PBS-EDTA solution in step 7) is an EDTA mixed solution at pH 7.2-7.4 containing 0.005-0.05M PBS and a final concentration of 2-3 mM EDTA; the PBS solution is 0.005-0.05M PBS solution at pH 7.2-7.4; alternatively, the PBS buffer solution can contain calf serum and/or EDTA, and the concentration of calf serum or EDTA can be the concentration commonly used in the art.

In the method of the present application, as one of the embodiments, the centrifuging conditions in step 7) are 1500-2900 rpm for 5-10 min.

As one of the embodiments, the method of the present application includes

(I) Isolation of Human Peripheral Blood Mononuclear Cells from Peripheral Blood

(1) 0.2 mL of the isolated original blood sample was taken for counting, and 1 mL of the sample was retained with labeled information and stored at 4° C.;

(2) centrifuging the blood in a centrifuge tube at 1500-2900 rpm for 15-20 minutes, and storing the upper plasma in a 2 ml cryopreservation tube at −80° C.;

(3) diluting the cell precipitate of the rest of the blood with PBS solution by a volume ratio of 1:1;

(4) prepare a 15 ml centrifuge tube and fill the pipette with lymphocyte separation solution equal to the blood cell diluent;

(5) slowly add the blood cell diluent to the centrifuge tube in step (4), keeping the separation fluid well stratified;

(6) slowly placing the centrifuge tube into a low-speed centrifuge, balancing, and centrifuging at 1500-2900 rpm for 15-20 mins;

(7) after centrifugation, the upper plasma was slowly absorbed with a 10 ml pipette or a 3 ml Pasteur pipette and discarded;

(8) pipetting middle buffy coat cells by circles into a new 15 ml centrifuge tube, adding PBS solution to a volume of 10 ml, counting 200 μl of cell suspension on a whole blood cell counter for subsequent adjustment of cell concentration, and centrifuging the rest of the cell suspension at 1500-2900 rpm for 5-10 mins; and

(9) centrifuging, discarding the supernatant, and, according to the result of counting, adding appropriate amount of pre-cooled PBS solution at 2-8° C. to adjust the cell density to 1.5-2.0×10⁶/ml.

As an example, the concentration can be 1.5×10⁶/ml, 1.6×10⁶/ml, 1.7×10⁶/ml, 1.8×10⁶/ml, 1.9×10⁶/ml or 2.0×10⁶/ml.

(II) Preparation of Flow Cytometric Samples

(10) sample staining

Each of the flow tubes is added with 100 μl cell suspension, and added with flow cytometric antibody to label lymphocyte (it is noted to arrange negative cell tube, ISO tube and single positive tube), gently shook in a vortex mixer to evenly mix the antibody with cells, and incubated in the dark at 2-8° C. for 10-30 minutes, preferably 15-20 minutes and most preferably 20 minutes.

(11) sample washing and solution adding

After incubating, the cells are resuspended in 2 ml PBS solution at 2-8° C., and centrifuged at 1500-2900 rpm for 5-10 min. The supernatant is discarded.

\textcircled{1} If a detection on a flow cytometer is performed immediately, the cells are mixed with 400 μl pre-cooled PBS-EDTA solution ready for flow cytometric detection;

\textcircled{2} If the detection cannot be performed immediately, cells are resuspended in 500 μl 0.05-5% formalin, vertex mixed, and stored at 4° C. in the dark. Before detection, each of the tubes is added with 2 ml PBS solution, centrifuged at 1500-2900 rpm for 5-10 min to discard the supernatant, and added with 4041 pre-cooled PBS-EDTA solution to evenly mix cells for flow cytometric detection.

By using the method of the present application, the lymphocyte sample can be easily obtained and quantified, and the detection of all detection indexes can be realized only by a small amount of in vitro peripheral blood samples; at the same time, it can realize the detection of cells including but not limited to DC cells (dendritic cells) and the like.

The specific experimental steps of the detection method of the present application are divided into four steps: acquisition of in vitro blood sample, cell separation, antibody incubation and flow cytometric detection. Based on the principle of antigen-antibody binding, the present application adopts antibodies combined with different fluorescence to bind specific antigens on the surface of cells, so as to label such subpopulations.

The proportion and number of such cells can be analyzed during flow cytometric detection.

Acquisition of in vitro blood samples: obtained by collecting peripheral blood from blood collection vessels in vitro.

Cell separation: the blood sample separation method of the present application can save more material cost and time cost, and provide faster and more accurate experimental results.

In a verified experimental method, the peripheral blood is separated and washed with lymphocyte separation solution, diluted to the specified range of concentration, and stored at 4° C. for use.

Antibody incubation: fluorescent antibody corresponding to specific antigen on the surface of each type of cells were selected; and the isolated cells were diluted to an appropriate concentration, incubated according to the verified concentration of fluorescent antibody and verified time, then washed with washing solution to remove excessive antibody, and mixed with flow cytometric buffer solution.

Flow cytometric detection: a flow cytometer is used for detection and analysis.

The experimental method of the present application is different from the traditional flow cytometric method. The present application highly enriches lymphocyte, and the cells that affect the detection results such as red blood cells and granulocytes are removed, so that many cell subpopulations with low expression or trace amount of expression can be clearly presented, providing obvious populations and facilitating analysis and research. From another point of view, by using high purity lymphocyte separated by the present application, the efficiency of binding antibody per unit volume will be much higher, and more than half of the antibody cost and antibody incubation time can be saved.

Compared with the prior art, the present application has the following advantages: 1. The lymphocyte is separated and enriched by using the lymphocyte separation solution, which ensures reliable and accurate detection results and save antibodies and reagents; 2. By detecting enriched lymphocyte subpopulations, it can effectively analyze lymphocyte subpopulations with small number of cells and ensure effectively and reasonably staining the lymphocyte subpopulations as defined; 3 The lymphocyte subpopulations that can be detected by the method of the present application cover an immune recognition early-warning system, cellular immune system, humoral immune system and non-specific immune cell system, so the detection results can comprehensively reflect the state of the lymphocyte system; 4. The present application can clearly define two subtypes of DC cells; 5. The invention can comprehensively analyze and detect the lymphocyte subpopulations, providing a scientific basis for comprehensive understanding of the immune status.

### BRIEF DESCRIPTION OF DRAWINGS

FIG. 1: detection diagram of lymphocyte subsets in Example 2;

FIG. 2: detection diagram of lymphocyte subsets in Example 2;

FIG. 3: detection diagram of lymphocyte subsets in Example 3;

FIG. 4: detection diagram of different lymphocyte subsets in example 3;

FIG. 5: detection diagram of DC cell subtypes in Example 3;

FIG. 6: comparison of cell populations of the sample obtained by the method of Example 1 and the sample obtained by the lysis method in the literature in Example 4; (the upper three figures show the flow cytometric diagrams obtained by the lysis method, and the lower three figures show the flow cytometric diagrams obtained by separation of corresponding samples in the upper three figures.)

FIG. 7: comparison diagram of cell enrichment between the sample obtained by the method of Example 1 and the sample obtained by the lysis method in the literature in Example 4;

FIG. 8: comparison diagram of enrichment of different types of cells between the sample obtained by the method of Example 1 and the sample obtained by the lysis method of literature in Example 4.

### DETAILED DESCRIPTION

The following embodiments and test examples are used to further elaborate the application, not intended to limit the effective scope of the present application in any way.

### Example 1 Preparation of PBMC Samples Needed for Flow Cytometry

1. Purpose

preparing PBMC samples needed for flow cytometric detection (also referred to as “samples” in the context of the present application)

2. Principle

There are relatively complete antigens or receptors remained on the surface of living cells. Fluorescent labeled antibodies are used to bind to corresponding antigens on the surface of cells, and fluorescence intensity and positive percentage are measured to provide the density and distribution of corresponding antigens.

3. Preparation of Instruments and Consumable Materials

3.1 Instruments

Super Clear Workbench, Era Beili low speed centrifuge DT5-4, a vortex mixer, pipettes of various ranges, and a blood cell counter

3.2 Consumable Materials

15 ml centrifuge tube, 10 ml pipette, 1.5 ml centrifuge tube, a flow tube, 3 ml pasteurizer pipette, and pipette tips of various ranges.

3.3 Formulations of Reagents

| Name | Stock solution | Formulation |
|---|---|---|
| 1× PBS | 10× PBS | stock solution: purified water = 1:9 |
| 0.1% formalin | 10% formalin | stock solution:1× PBS = 1:9 |
| PBS solution | 1× PBS | 1× PBS (0.01M PBS, pH = 7.2-7.4) |
| 1× hemolysin | 10× hemolysin | stock solution: purified water = 1:9 |
| PBS-EDTA | 1× PBS | adding 0.5M EDTA to 1× |
| solution | | PBS (0.01M |
| | | PBS; pH = 7.2-7.4) until the final |
| | | concentration of EDTA is 2.5 mM |

Routine flow cytometric antibodies;

4. Operation Procedure (Illustrated by Taking 5 ml In Vitro Original Blood Sample as an Example)

4.1. Separation of In Vitro Peripheral Blood Sample (PBMC)

(1) obtaining 0.2 ml in vitro original blood sample and counting;

(2) adding 5 ml blood to a 15 ml centrifuge tube, centrifuging at 1500-2900 rpm for 15-20 min, and discarding upper plasma;

(3) diluting cell precipitate of the rest of the original blood with PBS solution by volume;

(4) providing a centrifuge tube and adding lymphocyte separation solution having a volume equal to the volume of the blood cell diluent to the centrifuge tube by using a pipette;

(5) adding blood cell diluent into the centrifuge tube in step (4) slowly for keeping the layer of the separation solution clear;

(6) slowly placing the centrifuge tube into a centrifuge, balancing, and centrifuging at 1500-2900 rpm for 15-20 min;

(7) slowly suctioning and discarding the supernatant with a pipette after centrifuging;

(8) pipetting middle buffy coat cells into a new centrifuge tube, adding PBS solution to a volume of 10 ml, counting 200 μl of the cell suspension on a blood cell counter, and centrifuging the rest of the cell suspension at 1500-2900 rpm for 5-10 min;

(9) discarding the supernatant after centrifuging, and removing residual liquid by using a pipette. According to the result of counting, appropriate amount of pre-cooled PBS solution at 4° C. was added, so as to adjust the density of cells to 1.5-3.0×10⁶/ml, for example, 2×10⁶/ml.

4.2 Preparation of flow cytometric samples (the temperature was controlled in this operation, and the samples and antibodies were operated in an ice box throughout the operation)

(1) Sample Staining

A flow tube was added with 100 μl lymphocyte suspension, added with flow cytometric antibody, gently shook in a vortex mixer to fully mix the antibody with the cells, and incubated in the dark at 2-8° C. for 20 minutes.

(3) Sample Washing

After incubating, the supernatant was discarded, and the cells were resuspended in 2 ml pre-cooled PBS solution, and centrifuged at 1500-2900 rpm for 5-10 minutes.

(4) Completion of Samples

\textcircled{1} If the detection on a flow cytometer is performed immediately, the supernatant is discarded and the cells are mixed in 400 μl pre-cooled PBS-EDTA solution for flow cytometric detection;

\textcircled{2} If the detection cannot be performed immediately, cells are suspended in 500 μl 0.05-5% formalin, vortex mixed, and left to stand at 4° C. in the dark. Before detection, each of the tubes is added with 2 ml PBS solution, and centrifuged at 1500-2900 rpm for 5-10 minutes to discard the supernatant, and the cells are mixed with 400 μl pre-cooled PBS-EDTA solution for detection on a flow cytometer.

5. Notes

5.1 the antibodies and cells should be kept at low temperature (4° C.) during the operation.

5.2 after adding the antibodies, the antibodies and the cells should be fully mixed.

### Example 2 Detection of PBMC Samples

2.1 Instruments, Reagents and Samples:

Sample: prepared by the method of Example 1 from in vitro blood samples;

Flow cytometer: ACEA NovoCyte

2.2 Detection Method:

a) checking the status of the cytometer before starting up;

b) warming up the flow cytometer system for 5-10 minutes and performing perfusion (for about 15 minutes);

c) performing quality control procedure and proceeding to the next step after qualification;

d) setting the parameters of the flow cytometer, adjusting the voltage of each of detection channels, and setting the liquid flow speed to 35-40 μL/min;

e) vortex vibrating the prepared flow cytometric samples for 3 seconds, and subjecting the samples to detection under the conditions of: 15000 cells in the gate; and collecting samples.

2.3 Results of Detection

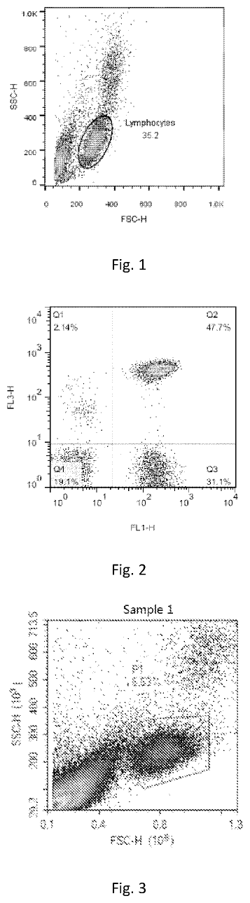

It can be seen from FIG. 1 that, in the plot of FSC vs SSC, gate 1: lymphocyte population, lymphocyte subpopulations can be clearly identified, and the data will be more reliable and accurate since it is populations of cells that are main analyzed.

It can be seen from FIG. 2 that: (1) gate 1 is selected to display FL1 and FL3 area in gate 1; (2) select cross gate 2 in the gate 1: CD3+CD4+ cell population (Q2 region) is selected. The lymphocyte subpopulations to be analyzed are well separated, the gate is more easier to set, and the data is more accurate.

### Example 3 Detection of PBMC Samples

3.1 Instruments, Reagents and Samples:

Sample: prepared by the method of Example 1 from 5 ml in vitro blood samples; Flow cytometer: ACEA NovoCyte

3.2 Detection Method:

a) checking the status of the cytometer before starting up;

b) warming up the flow cytometer system for 5-10 minutes and performing perfusion (for about 15 minutes);

c) performing quality control procedure and proceeding to the next step after qualification;

d) setting the parameters of the flow cytometer, adjusting the voltage of each of detection channels, and setting the liquid flow speed to 35-40 μL/min;

e) vortex vibrating the prepared flow cytometric samples, and subjecting the samples to detection under the conditions of: 15000 cells in the gate; and collecting samples.

3.3 Results of Detection

1) FIG. 3 shows the analysis of lymphocyte subpopulations obtained from the peripheral blood of one healthy person by lymphocyte separation method, in which a population of lymphocyte is in circled gate P1. As can be seen from FIG. 3, the background is clean, and the population of lymphocyte and population of cell debris are well and ideally separated.

It can be seen that, the present application separates and enriches lymphocytes by using lymphocyte separation solution, which are obviously divided, ensuring reliable and accurate detection results while reducing antibodies and reagents.

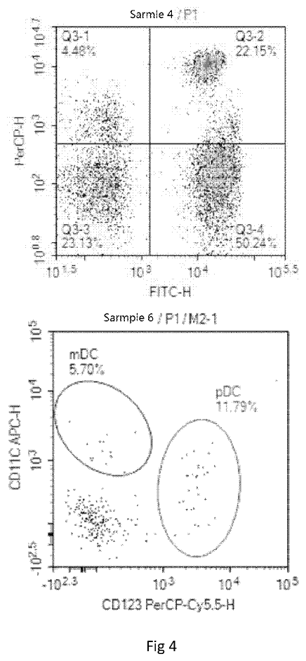

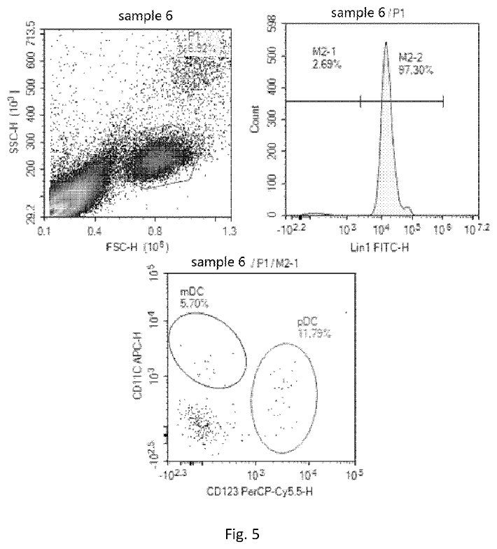

2) FIG. 4 shows that, through the detection of enriched lymphocyte subpopulations, lymphocyte subpopulations with relatively small number of cells are effectively analyzed, and also the specific lymphocyte subpopulations can be effectively stained; In FIG. 4, for sample 4 on the left, Q3-2 is CD8+T cells, which are obviously separated via separation and enrichment so that the subpopulations can be well analyzed. The right figure of FIG. 4 shows the cell subpopulations with a small proportion of lymphocyte subpopulations, that is, DC cell subpopulation, which can also be accurately analyzed after enrichment.

3) FIG. 5 clearly defines two subtypes of DC cells, which otherwise should not be identified in conventional lymphocyte subpopulation analysis. The two subtypes of cells were enriched by separation. As shown in the right 1 of FIG. 5, the population is obviously separated and the boundary is clear.

### Example 4 Comparison Between the Sample Prepared in the Method of the Present Application and the Sample Obtained by the Hemolysin Lysis Method

4.1 Samples and Reagents:

The lymphocyte sample of the present application: prepared by the method of Example 1 from in vitro peripheral blood sample;

Comparative lymphocyte samples: prepared by the method according to following section 4.2.1;

5 ml anticoagulant blood sample;

1×PBS: prepared from 10×PBS solution: pure water=1:9 by volume;

PBS solution: 1×PBS;

PBS-EDTA solution: prepared by adding 0.5m EDTA to 1×PBS until the final concentration of EDTA is 2.5 mM;

Conventional flow cytometric antibodies: anti-CD3 antibody, lin1[Lineage cocktail 1], anti-CD123 antibody, anti-CD11 antibody, CD3 FITC, CD8PerCP, CD4PerCP.

4.2 Testing Method:

4.2.1 Hemolysin Lysis Method:

(1) Sample staining: adding 100 μl anticoagulant blood sample into each of flow tubes, and adding flow cytometric antibody to label lymphocyte subpopulations respectively, so that the antibody was fully mixed with cells, and incubating the flow tubes in the dark at 2-8° C. for 20 minutes.

(2) Lysis: adding 2 ml 1×BD hemolysin (i.e. red blood cell lysis solution) to the flow tube, and performing lysis for 5 minutes.

(3) Sample washing: 5 minutes later, centrifuging at 1500-2900 rpm for 10 minutes, discarding the supernatant, adding 2 ml pre-cooled PBS solution to resuspend the cells, centrifuging at 1500-2000 rpm for 5-10 minutes, discarding the supernatant, adding 400 μL PBS-EDTA solution at 2-8° C., and evenly mixing.

4.2.2 Method for Detection on a Flow Cytometer

a) before starting up, checking the volume of sheath barrel, waste liquid barrel, detergent and flushing agent, adding enough sheath liquid, and pouring out waste liquid;

b) warmed up the flow cytometric system for 5-10 minutes and perform perfusion (for about 15 minutes);

c) performing quality control procedure and proceeding to the next step after qualification;

d) setting the parameters of the flow cytometer, adjusting the voltage of each of detection channels, and setting the liquid flow speed to 35-40 μL/min;

e) vortex vibrating the prepared flow cytometric samples of the present application and comparative lymphocyte samples for 3 seconds, respectively, and subjecting the samples to detection under the conditions of: 15000 cells in the gate; and collecting samples.

4.3 Results of Experiments:

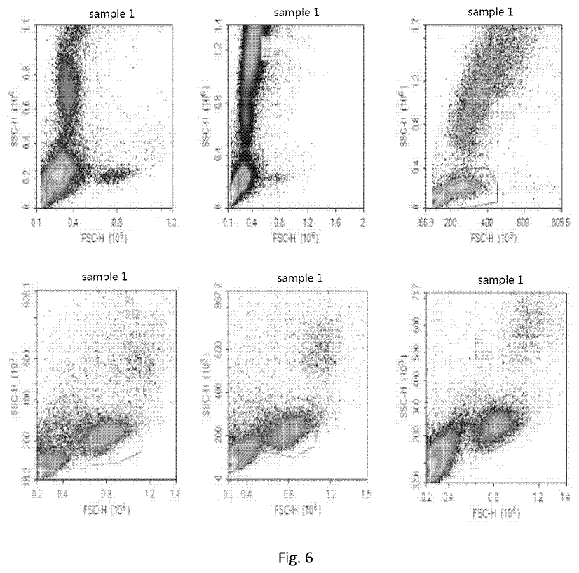

4.3.1 through comparison, it can be seen that there is much debris in the sample obtained by lysis, and the background is not clean, which influences the collection and analysis of flow cytometry data, and the compensation adjustment of lymphocytes may cause bias phenomenon; and the present method (method by using lymphocyte separation solution) provides clean background and clear and obvious populations (see FIG. 6).

In particular, for the lymphocytes in the gate circled in the lymphocyte subpopulation analysis obtained by lysis method, it can be seen from the figure that the lymphocyte subpopulation and the cell debris population is not ideally separated (see the three figures in the first row of FIG. 6).

For the lymphocytes in the gate circled in the lymphocyte subpopulation analysis obtained by the present method, it can be seen for the figure that the lymphocyte subpopulations and cell debris population are well and ideally separated (see the third figures in the second row of FIG. 6).

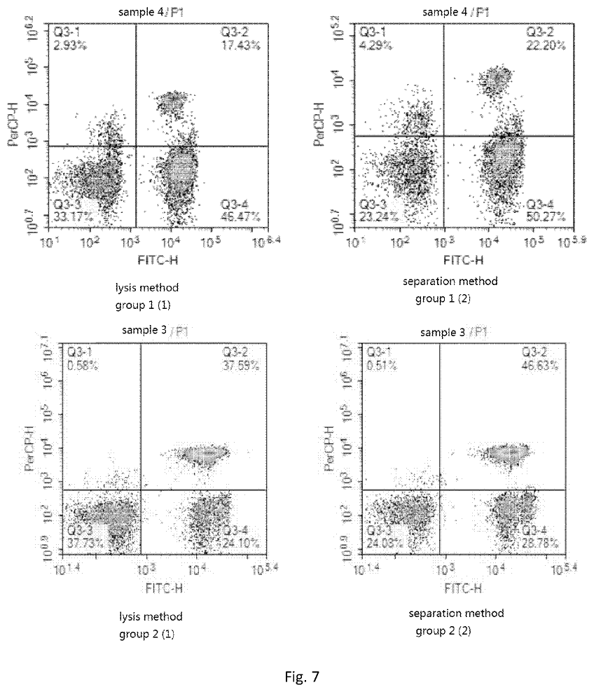

4.3.2 It can be seen from FIG. 7 that, in the present method, when the fluorescent antibody is used, since the number of lymphocytes is counted and the fluorescent antibody is added in proportion, more than 70% of the antibody is saved. The amount of combined antibody used in the present application is 15 μL, which is 60 μL in the lysis method. The method of the present application does not cause excessive or insufficient antibody. It can be seen from FIG. 7 that CD3 FITC and CD8PerCP were labeled in group 1, and CD3 FITC and CD4PerCP were labeled in group 2. Due to the different samples obtained by different treatment methods, for the same separated peripheral blood sample, the result showed that, the proportion of double positive groups in the sample obtained by the lysis method is significantly reduced, and the amount of antibody cannot be quantified; while for the sample obtained by the method of the present application, the antibody can be quantitatively added according to the amount of cells. In addition, it is obvious that the cells obtained by the separation method are significantly enriched.

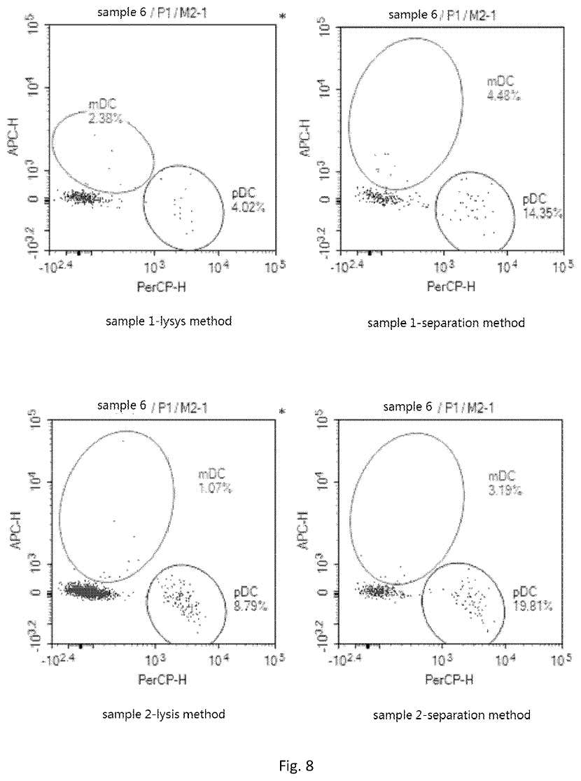

4.3.3 it can be seen from FIG. 8 that the sample prepared by the present application has enrichment effect for the sample with small proportion of cell subpopulations. For the same separated peripheral blood sample, for the MDC and PDC accounting for small proportion, the observation results of the samples as obtained are different in the lysis method and the method of the present application, and the cell subpopulations in the samples obtained by the present application are enriched, which can be shown from the number and percentage of cells.

1. A method for detecting lymphocytes in immune cells, comprising the steps of:
a) lymphocyte samples stained with immunofluorescence antibody were added into the pre-cooled PBS-EDTA solution, and the cells were mixed and prepared for flow cytometry detection;
b) starting up and warming up a flow cytometer system, adjusting the flow speed to a low-speed range of 10-29 μl/min or a medium speed range of 30-44 μl/min, then adding PBS-EDTA solution into a sample tube, and flushing a nozzle system of liquid stream;
c) the sample of homogenous cells obtained in step a) was added to the sample tube and then tested.

2. The method according to claim 1, wherein the PBS-EDTA solution is a mixed solution containing 0.005-0.05M PBS and a final concentration of 2-3 mM EDTA with a pH of 7.2-7.4.

3. The method according to claim 1, wherein step a) further includes: the concentration of cells in the lymphocyte sample is 1×10⁶-10×10⁶/ml.

4. The method according to claim 1, characterized in that, step a) further includes: the volume of the lymphocyte sample is 50-200 μl.

5. The method according to claim 1, wherein in step b), the flow speed is adjusted to a medium speed range of 30-44 μl/min; preferably a medium speed range of 35-40 μl/min.

6. The method according to claim 1, wherein step b) further includes a preheating time of 5-10 minutes.

7. The method according to claim 1, wherein step c) further includes setting conditions of 3000-20000 cells in the gate, preferably 15000 cells in the gate, and collecting samples.

8. The method according to claim 1, wherein, the flow cytometric cell sample is prepared by the following method:
1) in vitro anticoagulant blood samples were taken for centrifugation to separate plasma and blood cell precipitation to obtain blood cell precipitation;
2) blood cell precipitation was diluted with PBS solution to obtain blood cell diluent;
3) the blood cell diluent was slowly added to a centrifuge tube containing an equal volume of lymphocyte separation solution, and then centrifuged to remove the plasma to obtain the middle buffy coat cells diluent;
4) adding the buffy coat cells obtained in step 3) into a centrifuge tube, adding PBS solution under stirring to obtain a suspension, centrifuging, removing supernatant, and then adding PBS solution to adjust the cell concentration to 1×10⁵/ml-1×10⁸/ml, preferably 1.5×10⁶/ml-5×10⁶/ml, further preferably 1.5×10⁶/ml-3.0×10⁶/ml;
5) adding the lymphocyte cells obtained in step 4) after adjusting the concentration thereof into a flow tube; and adding flow cytometric antibody to label lymphocyte subpopulations, evenly mixing, standing at 2-8° C. and incubating without light;
6) adding PBS solution, mixing, and centrifuging to remove unbound antibody;
7) completion of samples
\textcircled{1} If a detection on a flow cytometer is performed immediately, the supernatant is discarded and the cells are evenly mixed with PBS-EDTA solution pre-cooled to 2-8° C. for flow cytometric detection;
\textcircled{2} If the detection cannot be performed immediately, cells are directly resuspended by adding 0.05-5% formalin, mixed, and left to stand at 2-8° C. in the dark; and before detection, each of the tubes is added with PBS solution, centrifuged to discard the supernatant, and added with PBS-EDTA solution at 2-8° C. to evenly mix cells for flow cytometric detection.

9. The method according to claim 8, wherein the PBS solution is 0.005-0.05M PBS solution at pH 7.2-7.4; and the PBS-EDTA solution is a mixed solution at pH 7.2-7.4 containing 0.005-0.05M PBS and a final concentration of 2-3 mM EDTA.

10. The method according to claim 8, wherein step 1) further comprises centrifuging conditions of 1500-3500 rpm for 5-30 min, preferably 1500-2900 rpm for 15-20 min.

11. The method according to claim 8, wherein step 2) further comprises diluting blood cell precipitate with PBS solution by a volume ratio of 1:0.5 to 1:2, preferably 1:1.

12. The method according to claim 8, wherein step 3) further comprises centrifuging conditions of 1500-3500 rpm, 10-20 min and 4° C.; preferably, 1500-2900 rpm, 15-20 min and 4° C.

13. The method according to claim 8, wherein step 4) further comprises centrifuging conditions of 1500-3500 rpm, 5-20 min and 4° C.; preferably centrifuging conditions of 1500-2900 rpm, 5-10 min and 4° C.

14. The method according to claim 8, wherein PBS solution is added to the centrifuge tube in step 4) until the volume is 10 ml-15 ml.

15. The method according to claim 8, wherein step 6) further comprises centrifuging conditions of 1500-2900 rpm for 5-10 min.

16. The method according to claim 8, wherein the amount of PBS solution added in step 6) is 2 ml.

17. The method according to claim 8, wherein the PBS-EDTA solution in step 7) is an EDTA mixed solution at pH 7.2-7.4 containing 0.005-0.05M PBS and a final concentration of 2-3 mM EDTA; and the PBS solution is 0.005-0.05M PBS solution at pH 7.2-7.4.

18. The method according to claim 8, wherein the centrifuging conditions in step 7) are 1500-2900 rpm for 5-10 min.

19. The method according to claim 8, wherein the method comprises:
(I) Isolation of Human Peripheral Blood Mononuclear Cells from Peripheral Blood
(1) 0.2 mL of the isolated original blood sample was taken for counting, and 1 mL of the sample was retained with labeled information and stored at 4° C.;
(2) centrifuging the blood in a centrifuge tube at 1500-2900 rpm for 15-20 minutes, and storing the upper plasma in a 2 ml cryopreservation tube at −80° C.;
(3) diluting the cell precipitate of the rest of the blood with PBS solution by a volume ratio of 1:1;
(4) prepare a 15 ml centrifuge tube and fill the pipette with lymphocyte separation solution equal to the blood cell diluent;
(5) slowly add the blood cell diluent to the centrifuge tube in step (4), keeping the separation fluid well stratified;
(6) placing the centrifuge tube into a low-speed centrifuge, balancing, and centrifuging at 1500-2900 rpm for 15-20 mins;
(7) after centrifugation, the upper plasma was slowly absorbed with a 10 ml pipette or a 3 ml Pasteur pipette and discarded;
(8) pipetting middle buffy coat cells by circles into a new 15 ml centrifuge tube, adding PBS solution to a volume of 10 ml, counting 200 μl of cell suspension on a whole blood cell counter for subsequent adjustment of cell concentration, and centrifuging the rest of the cell suspension at 1500-2900 rpm for 5-10 mins; and
(9) centrifuging, discarding the supernatant, and, according to the result of counting, adding appropriate amount of pre-cooled PBS solution at 2-8° C. to adjust the cell density to 1.5-2.0×10⁶/ml;
(II) Preparation of flow cytometric samples
(10) sample staining
Each of the flow tubes is added with 100 μl cell suspension, added with corresponding flow cytometric antibody so that the antibody is evenly mixed with cells, and incubated in the dark at 4° C. for 10-30 minutes, preferably 15-20 minutes and most preferably 20 minutes.
(11) sample washing and solution adding
After centrifuging, the supernatant is discarded, and the cells are resuspended in 2 ml PBS solution at 2-8° C., and centrifuged at 1500-2900 rpm for 5-10 min to discard supernatant after centrifuging;
\textcircled{1} If a detection on a flow cytometer is performed immediately, the cells are evenly mixed with 400 μl pre-cooled PBS-EDTA solution ready for flow cytometric detection;
\textcircled{2} If the detection cannot be performed immediately, cells are resuspended in 500 μl 0.05-5% formalin, vertex mixed, and stored at 4° C. in the dark; and before detection, each of the tubes is added with 2 ml PBS solution, centrifuged at 1500-2900 rpm for 5-10 min to discard the supernatant, and added with 400 μl pre-cooled PBS-EDTA solution to evenly mix cells for flow cytometric detection.
